(19) United States
(12) Patent Application Publication
(10) Pub. No.: US 2007/0038137 A1
(43) Pub. Date: Feb. 15, 2007

(54) Cardio-function cafeteria system and methodology

(75) Inventors: Patricia A. Arand, McMinnville, OR (US); Robert A. Warner, Tigard, OR (US); Peter T. Bauer, West Linn, OR (US)

Correspondence Address: ROBERT D. VARITZ, P.C., 4915 SE 33RD PLACE, PORTLAND, OR, 97202, US

(73) Assignee: Inovise Medical, Inc.

(21) Appl. No.: 11/442,467

(22) Filed: May 25, 2006

Related U.S. Application Data
(60) Provisional application No. 60/685,316, filed on May 26, 2005.

(51) Int. Cl. A61B 5/04

(52) U.S. Cl. 600/509

(57) ABSTRACT

A computer-based cafeteria system, accessible through a user interface, and a related methodology, for gathering, handling, observing, presenting and addressing cardio-function data from a selected patient. The methodology-implementing system includes: (a) structure for collecting, over extended time, plural-heartbeat, cardio-function data, including ECG and heart-sound data; (b) a computer connected to the collecting structure for receiving and associating selected elements of collected data so as to offer, via user-selection, a display output potentially including (a) graphical, numeric, and textual information, and (b) blends thereof, an opportunity to assess the presence and character of a cardio-function condition of the subject patient's heart, including especially the detectable presence of cardio-function trend behavior of that heart useable to implement constructive cardio-function intervention; and (c) display output structure connected to the computer for presenting an assessment-relevant cardio-function-condition display (a) of such associated data, (b) of selected elements thereof, and (c) of detected cardio-function trend behavior.

### CROSS REFERENCE TO RELATED APPLICATION

This application claims priority to currently co-pending U.S. Provisional Patent Application Ser. No. 60/685,316, filed May 26, 2005 for “ECG/Sound Real-Time Monitoring System, and Related Methodology, With Selectable, Interrelated, Plural-Facet Screen Display”. The entire disclosure content of that provisional case is hereby incorporated herein by reference.

### BACKGROUND AND SUMMARY OF THE INVENTION

As is true in many areas of practically useful knowledge, the field of cardiology is rich today with innovation. The present invention—system and methodology—utilizing sophisticated computer algorithmic and visual-display technologies, engages this field, and offers a dramatic advance in the capability for understanding heart disease in many of its illusive nooks and crannies, and for promoting quick and accurate diagnoses of the roots of many heart (cardio-function) problems, even to the point of offering constructive intervention, or a least system-user-encouraged constructive intervention, in the applications of “treatments” for these problems.

As will be seen, this invention focuses not so much, or even at all particularly, on detecting an emergency, cardio-alarm-type situation. Rather, and as will be further explained below, it is focused on collecting time-extended, current-state-of-a-patient's-heart, cardio-relevant data principally to detect, through observing potentially device- or drug-therapy-affectable, cardio-functionality trends (for example an S3 heart sound trends over the chosen observation time), emerging, or existing, non-alarm cardio conditions which may then, on-the-spot, be addressed constructively and correctively by appropriate “feedback actions and activities”. Such on-the-spot “addressing” may be performed automatically by the system and methodology of the present invention, or may be intelligently addressed by a well-informed system user who is provided, by richly detailed display-output information, including important correlation information, and algorithmically analyzed information, furnished by the invention, with a powerful guide toward selecting and implementing an appropriate cardio-functionality improvement approach for each particular patient.

According to the invention, a relatively large plurality of real-time ECG and heart-produced acoustic signals are gathered over extended time from a subject patient. Other kinds of data, such as blood pressure and pulse oximetry data, may also simultaneously be gathered.

These signals and are fed to a digital computer which is armed with cardio-interpretive algorithms, and coupled preferably to one, or both, a hard-copy and/or a screen-virtual visual output-display structure(s). These output-display structures, such as printers and electronic screen-display devices, sit poised to present, under computer control, various informative and intuitive output displays, including basic waveform displays, waveform snippet displays, waveform correlation displays, numeric and textual displays, and all of these (and more) being presentable selectively with or without associated, computer-intelligence-based, cardio-function analysis(es)/assessment(s).

A user interface included in the system, preferably a screen-borne virtual interface which is unified with a screen-virtual output display, allows a trained user, such as a doctor or other kind of clinician, to select what kinds and contents of cardio-function display outputs are to be computer-created from incoming patient data, and the extent to which such outputs will be presented with (a) no, (b) some, or (c) much computer-performed analysis/assessment and “judgment calling”. Minute details of relevant cardio-functionality evidenced in the incoming patient data are found when requested, and are made selectively “output viewable”.

A “system-connected” subject patient may be “deployed” in a condition ready to receive, or to engage in, over time, different selected therapies, such as pacemaker input (device) therapy, drug input therapy, exercise (device or otherwise) therapy, and so on. The system user may also call for different types and styles of selected cardio-function trend displays during utilization of such therapies to observe trends in a subject patient's heart behavior as a function of selectively changed “applied therapy”. This powerful capability offers the important opportunity to “fine tune”, in real time, a subject patient's cardio-behavior so as to improve, or to enable improvement in, that behavior. The system computer may even be enabled to accomplish such “fine tuning” automatically. The striking and enormous utility of these last-mentioned capabilities will be immediately apparent to those skilled in the relevant art.

The proposed system of the present invention may be made to be extremely small (think laptop), and also made, therefore, to be highly portable for use in a variety of different, convenient settings. It may also be quite inexpensive in the overall scheme of cardio-relevant devices and methodologies.

These and various other features, advantages, and new and useful opportunities which are offered by the system and methodology of the present invention will become more fully apparent as the description which now follows below is read in conjunction with the accompanying drawings.

### DESCRIPTION OF THE DRAWINGS

FIG. 1 is a pictorial block/schematic diagram of a preferred embodiment of a system made in accordance with the present invention, which system implements the preferred methodology of the invention.

FIG. 2 is a block/schematic diagram illustrating the system and methodology of FIG. 1 in a slightly different degree of detail. This figure contains certain included textual information which helps to illustrate features of the invention, particularly with respect to user-enabled selectability which is associated with a user interface, and a display output.

FIG. 3 provides a graphical illustration, well known to those skilled in the art, of a parameter-legended illustration of various basic events of the usual left heart cardiac cycle. This figure carries labels of certain time intervals which are recognized to be useful to a physician or other clinician examining the cardio-condition of a patient's heart.

FIG. 4 is a graphical illustration which is similar in some respects to FIG. 3, in which input waveform data, including ECG and heart-produced acoustic data are shown on a common time scale, along with aortic pressure and left ventricular pressure, and with a pair of important time intervals, labeled QS1 (also known as EMAT) and LVST are indicated.

FIGS. 5-8, inclusive, present four different screen-borne display outputs, including a virtual user-interface, made available on a monitor-type display device included in the system of FIGS. 1 and 2. These outputs illustrate the versatility of the system and methodology of the present invention with regard to presenting useful cardio-function output displays, as follows: FIG. 5, plural ECG traces, or waveforms, corroborated with two heart-sound waveforms; FIG. 6, various ECG and heart-sound waveforms, sound waveform snippets, and heart-rate and blood pressure numeric data; FIG. 7, a split-screen display including ECG and heart-sound waveforms various, a pair of sound snippets, four illustrations of trend data, and heart-rate and blood pressure numeric data; and FIG. 8, another split-screen display which is similar to what is shown in FIG. 7.

FIG. 9 illustrates a printed output display including textual and numeric data, waveforms, sound and ECG snippets, trend data, and a bar-graph display.

FIG. 10 illustrates a pair of waveform snippets which may be selectively requested by a user of the system of the present invention to observe certain specific features that are present in cardio-relevant data collected from a patient.

FIG. 11 is a schematic diagram, partly graphical and partly block-like in nature, which illustrates several important different ways in which a user of the invention may call for, or otherwise utilize, the data-analysis and interpretation/assessment capabilities of the invention based upon both (a) correlation of patient input data per se, as well as (b) observed-trend behavior found in that data as, for example, when a control parameter, such as that of an interactive device (like a pacemaker), or a control therapy, is “applied” to a patient during operation of the system of the invention.

FIGS. 12, 13 and 14 each illustrates a slightly different output display based upon cardio-function trend data.

### DETAILED DESCRIPTION OF THE INVENTION

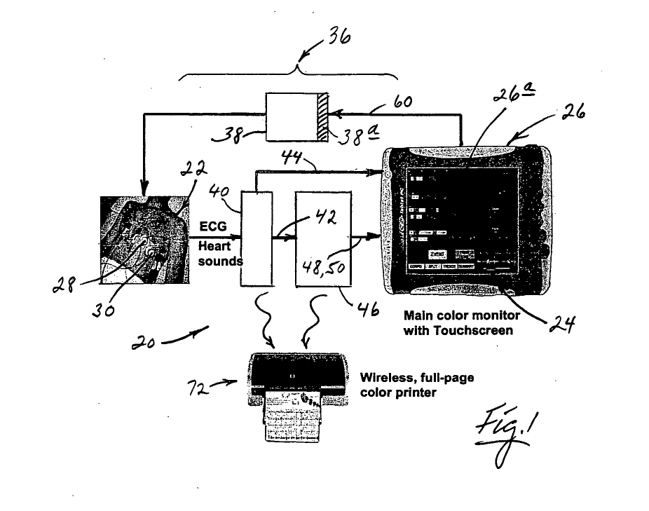

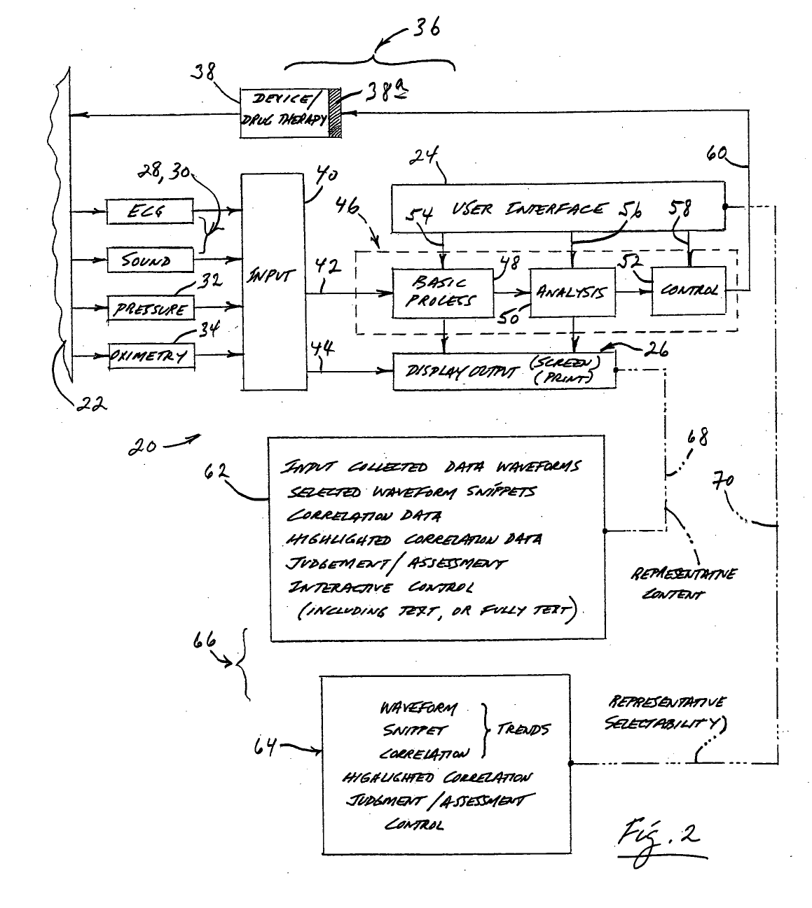

Beginning this description by looking at FIGS. 1 and 2, shown generally at 20 is a system which is being employed in real time to examine the heart condition (cardio-function or cardio-functionality) of a patient shown generally at 22 (pictorially in FIG. 1, and schematically in FIG. 2). System 20 is referred to herein as a cardio-function cafeteria system, and is made in accordance with a preferred embodiment of the present invention. System 20 implements a preferred form of the methodology proposed in accordance with the invention.

System 20, as shown in FIG. 1, is illustrated in a condition wherein it is only pictured collecting ECG and heart-produced acoustic signal information from patient 22. In FIG. 2, system 20 is illustrated in a condition gathering from patient 22 additional input signal information, as will shortly be explained. The term “cafeteria” is employed herein simply to reflect the fact that the system and methodology of this invention offer a great deal of versatility and choosability in the hands of a user with respect to the nature of data which is to be gathered, and the way, or ways, in which such gathered data is to be processed and output to, and by, a visual display device.

As those skilled in the art will quickly recognize, the drawing figures presented herein substantially fully explain both the structure and the methodology of the present invention in manners enabling ready and easy practice of all features of the invention.

With appropriate sensors coupled both to a patient, such as patient 22, and to system 20, time-extended (multiple heart-beat) cardio-relevant data input signals, of any design or category which relates to the functionality of at least one of these sensors, may be supplied to system 20 for the subsequent performance of the system's methodology—i.e., the methodology of the present invention. Having said that, it is important to recognize that practice of the invention substantially always includes the gathering of at least ECG and heart-produced acoustic signal data. All gathered signals are sent, after their collection, to an appropriately programmed digital computer which lies at the heart of system 20. More mention about this computer will be made shortly.

Regarding whatever particular data-collection sensors are chosen for use, a system user, via interaction with a system user interface which is coupled to the mentioned computer, may freely select the categories of system input data which are to be input and specifically utilized by system 20, recognizing, as mentioned above, that substantially always to be input the system are ECG and heart-produced acoustic data signals. In system 20, as pictured herein, the user interface employed is a display-screen virtual interface 24 which appears near the base of the display touchscreen 26a in a monitor-type electronic display output device 26. Interface 24 preferably includes a distribution of virtual control “buttons” made available for touching, or otherwise accessing, by a system user. In FIG. 1, user interface 24 is specifically shown adjacent the bottom of screen 26a (on the right side of FIG. 1), and in FIG. 2 is presented as a separate block in a schematic/block diagram. It should be understood that, while interface 24 herein takes the form of a touch-screen interface, other interface approaches, at least with respect to control, might include a keyboard, a mouse, or any other suitable form of user-input device.

ECG and heart-produced acoustic signals (data) are gathered preferably at the traditional V3 and V4 ECG sites by combined ECG and acoustic sensors, such as those shown at 28, 30, respectively, in FIG. 2. These sensors are, of course, interposed patient 22 and system 20. A preferred form of such a sensor, although others may be used if desired, is a device known as the Audicor® ECG and Sound Sensor made by Inovise Medical, Inc. in Portland, Oreg. Sensor 28 herein sits at the traditional V3 site, and sensor 30 at the traditional V4 site. In FIG. 2, two blocks “separated verbally” by the labels “ECG” and “SOUND” are shown, with a bracket utilized to indicate that these data-collecting categories are handled herein by a single, dual-function sensor device.

In addition to ECG and heart-produced acoustic data, non-exclusive, representative, other forms of relevant, gatherable and inputtable heart-useful data include blood pressure data (see block 32 in FIG. 2), and pulse oximetry data (see block 34 in FIG. 2). The two, relevant sensors which are associated with blocks 32, 34 in FIG. 2 are, of course, suitably interposed patient 22 and system 20.

As mentioned earlier herein, a central and extremely important feature of the invention is that it can be employed interactively in a feedback loop (see bracket 36 in FIGS. 1 and 2) which includes system 20, a patient, such as patient 22, data-collecting and inputting sensors, and some device, or some proposed, remedial therapy, such as a drug therapy (see block 38 in FIGS. 1 and 2) which can be made to “respond”, in an intervention mode, or modality, to system-analyzed, collected patient data, both to improve the information content of data gathered from the patient, and, very significantly, to intervene constructively and correctively to improve a patient's cardio-functionality. There are many illustrations of these interactive, intervening modalities, and block 38 is intended generally to provide surrogate representation for any one (or more) of devices/therapies, etc. which may implement any one (or more) of these modalities. Block 38 is illustrated herein with what is referred to as a control-parameter changer 38a.

A patient may, for example, be equipped with a change-parameter pacemaker whose specific function may be altered selectively by control signals sent to it to modify (and thereby improve) its working relationship with the heart—thus to enhance effective heart functionality. In such a pacemaker, the “change-parameter mechanism” may either (a) be directly on-board the pacemaker per se and remotely accessible in any suitable fashion, or (b) remotely located, as outside a patient's anatomy, and suitably “coupleable” to the basic, installed pacemaker hardware per se.

From another point of view, a change-application therapy may be employed, wherein a patient whose cardio-relevant data is being collected and analyzed is, under analyzed-data system control, given staged, controlled drug administrations aimed at affecting heart functionality.

So also may a patient be “stationed or deployed”, for example, on a treadmill, to provide stress-related, cardio-function data, with the system of the present invention, based upon analyzed and collected patient data, providing control signals to change treadmill operating parameters, such as traveling-belt speed, and/or inclination.

Other useful interactive devices/therapies will certainly come to the minds of those skilled in the art, and essentially any of these devices/therapies may readily be incorporated into practice of the present invention.

In a time-extended, real-time operation, as contemplated by the invention, as soon as a parameter-change control (device, therapy, etc.) “takes effect” on cardio-functionality, as will be evident, of course, in collected, cardio-relevant, patient data, the system and methodology of the invention, via an important “trend-observing” capability, enables a user immediately to observe the related effect on a patient's cardio-functionality, and then to use this observed information immediately, and extremely effectively and accurately, to provide immediate “fine tuning” of a patient's cardio system.

Continuing with a description of FIGS. 1 and 2, signals gathered from a patient are fed to an input signal-collection structure which is represented by a block 40. Within block 40, to the extent necessary, input signals, which are typically analogue signals, are converted to digital signals, and then fed, as indicated by arrows 42, 44, to slightly different locations resident within system 20. From the signal path indicated by arrow 42, signals are supplied to a digital computer 46 (previously generally mentioned) which performs all high-level signal processing, among other things, during practice of the methodology of the present invention. Computer 46 is also referred to herein as data-processing apparatus. Signals sent from block 40 as illustrated by arrow 44 are, effectively, fed directly to display output 26.

By use of user interface 24, a system user can call for the display of all or only some of input-gathered patient signals.

Signals supplied as illustrated by arrow 42 in FIGS. 1 and 2 to computer 46 flow, or may flow, therein to one or more of three computer-internal blocks seen at 48, 50, 52 in FIG. 2. User interface 24, as can be seen in FIG. 2, is effectively operatively connected to each one of blocks 48, 50, 52, as indicated by arrows 54, 56, 58, respectively, in this figure.

Under the control of user interface 24, and thus under the selective control of a system user, block 48 performs basic input signal processing, and allows a user selectively to call for presentations in a display output of different categories of signals, such as full waveform signals, selected waveform snippet signals, and time-based correlations of selected waveform, or waveform snippet, signals, and other things. More will be said about this practice shortly.

Block 50 in computer 46 is also referred to herein as prepared-intelligence, algorithmic, cardio-function analysis and interpretation structure. It is within this block, which incorporates what is referred to herein as cardio-condition-assessing algorithmic software, that certain very specialized signal processing takes place, at the selective call of the system user through user interface 24, to perform specialized data-analysis functions which are useful for presenting, in a display output, different specific kinds of cardio-relevant information, such as time-duration information, correlation-of-event information, detailed ECG information, acoustic “fingerprint” information (as described in U.S. Patent Application Publication No. 2006/0106322 A1, disclosing an invention entitled “Method and System Relating to Monitoring and Characterizing Heart Condition”), and so on. For disclosure enhancement purposes in this specification, the disclosure content of this just-mentioned publication is hereby incorporated herein by reference.

It is also within block 50 that, on selective call by a system user, computer-analyzed, detailed output information may be furnished to display output 26 in different categories of output, including output which shows correlated data without any indicated computer analysis or assessment, or similar output information accompanied by a performed computer assessment and judgment presentation. In other words, output in this category may offer a direct indication for a system user of what kind of condition, or conditions, appear(s) to be indicated by input data which has been processed within block 50. The user may also request various kinds of related numeric and textual output. A number of the drawing figures herein which are still to be discussed substantially illustrate this practice of the invention.

Block 52 in computer 46, under the selective call of a system user through interface 24, may directly supply output control signals, as over a line represented at 60 in FIGS. 1 and 2, to block 38 which forms part of previously mentioned feedback loop 36. As was pointed out earlier, this block 38 may represent a controllable pacemaker, or some other controllable machine, such as a treadmill, or it may represent a therapy, such as a drug administration therapy, all or any one of these to be associated with patient 22. Control signals coming from block 52 are supplied to the previously mentioned control-parameter changer which is represented by shaded sub-block 38a appearing within block 38 in both FIGS. 1 and 2.

As was mentioned earlier, in the particular form of system 20 now being described, user interface 24, in a sense, forms a portion of the display information which is provided on the touchscreen, 26a, in display output 26. In FIG. 2, two additional blocks 62, 64, which are linked by a bracket 66, represent, in included verbal outline form, the respective, high-level functionalities of user interface 24 and of display output 26. A dash-double-dot line 68 is shown connecting display output 26 with block 62 in FIG. 2, and a dash-triple-dot line 70 is similarly shown connecting user interface 24 with block 64 in FIG. 2. Bracket 66 is included in FIG. 2 to reflect the situation that user interface 24 is structured herein along with (as displayed on the touchscreen of) display output 26.

Listed in block 62, in high-level, textual outline form, are the several different key types of display output information, and information styles, which may selectively be presented by display output 26 on screen 26a, as called for by a system user through interface 24. Similarly, high-level outline text appearing in block 64 generally describes the wide range of selectability and signal-processing actions enabled for a system user through user interface 24.

As was mentioned earlier herein, another form of display output, or output device, might include a suitable form of printer structure, such as that which is shown as a wireless color printer generally at 72 in FIG. 1.

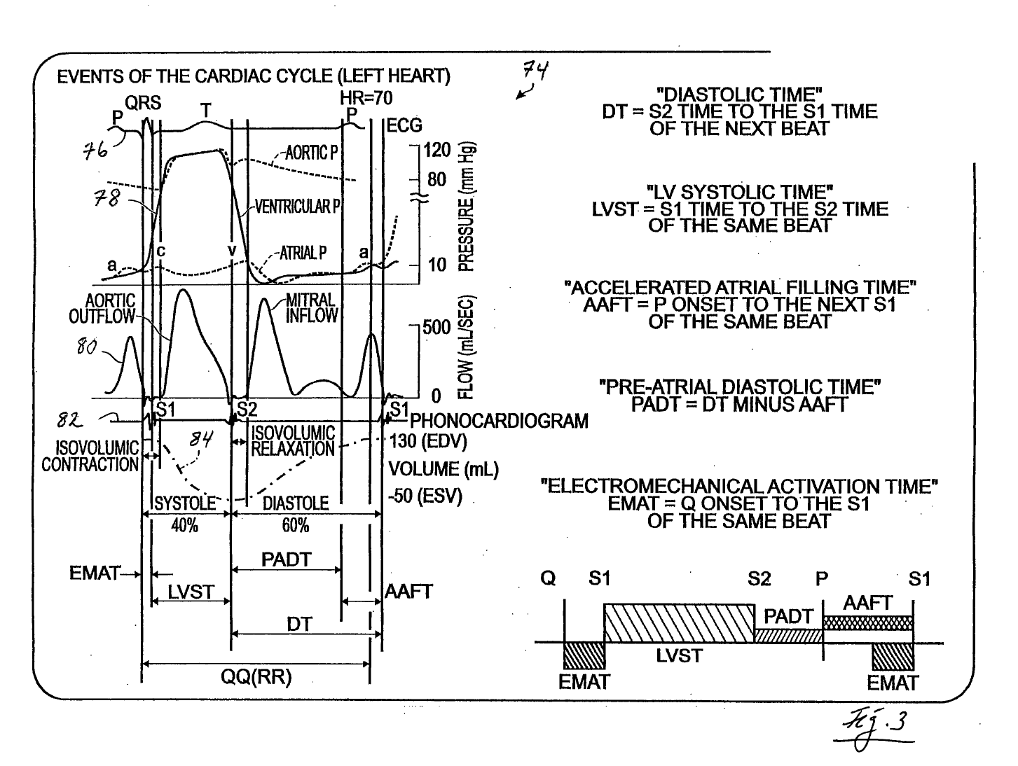

Turning now to all of the other drawings figures included herein, and generally describing (a) how system 20 functions, and (b) the kinds of information dealt with by this system, indicated generally at 74 in FIG. 3 is a relatively well known and conventional illustration of the events, and of certain measures of events, of a typical, single cardiac cycle with respect to the left side of the heart, referred to for simplicity purposes simply as the left heart. These events are those which normally take place, and are expected to take place, during such a cardiac cycle. At the lower portion of each of these two figures, certain timing intervals that are relevant in different ways to the information gathering and displaying practice of the present invention are specifically labeled by different clusters of capital letters. Set forth immediately below is a listing of the meanings of these letter labels:

AAFT—Accelerated Atrial Filling Time

DT—Diastolic Time

EMAT—Electromechanical Activation Time

LVST—LV Systolic Time

PADT—Pre-Atrial Diastolic Time

QQ(RR)—Interval between beats

Looking at, and describing, the time-based traces which appear in FIG. 3:
1. Top trace (76): ECG signal with makers for the p-wave (atrial depolarization), QRS complex (ventricular depolarization), and T-wave (ventricular re-polarization).
2. 2nd trace from top (78): Pressure tracings (obtainable through catheter measurements). Shown are the pressure curves for the left ventricle, the left atrium, and the aorta.
3. 3rd trace from top (80): Flow tracings (obtainable through echo tissue Doppler imaging (TDI)). When the pressure in the left ventricle is higher than in the aorta, blood is flowing into the aorta (happens between the S1 and S2 heart sounds)=aortic outflow; while the ventricular pressure is below the left atrial pressure, the left ventricle get filled with blood=mitral inflow. The mitral inflow occurs in three phases: a) passive filling (first hump between the S2 and the next S1)=Echo TDI E wave, b) diastasis (LA=LV pressure, small hump in the middle, hardly visible in Echo TDI), and c) active filling (atrial kick) while the left atrium contracts (hump just before the S1)=Echo TDI A-wave.
4. 4th trace from top (82): Heart sound trace showing an S1 (closure of mitral valve) and S2 (closure of aortic valve). The third heard sound is not shown, but it would occur toward the end of the E-wave in the flow trace.
5. 5th trace from top (84): Volume trace showing the changes in left ventricular volume from its minimum=ESV (end systolic volume, so the volume at the end of ventricular contraction) and its maximum=EDV (end diastolic volume, so the volume at the end of the ventricular filling phase).

FIG. 4 in the drawings, as was mentioned earlier herein, is somewhat similar to FIG. 3. FIG. 4 illustrates four categories of output-display-presentable, collectable patient data including, of course, ECG and heart-produced acoustical data, along with aortic pressure data and left ventricular pressure data. This figure also illustrates a pair of important time measurements, labeled QS1 and LVST, and also illustrating a clearly discernable presence of the so-called, well-recognized, third heart sound S3. What is shown in FIG. 4 might well take the form of a system-user-requested and selected correlation of waveform snippet data, on a common time base, illustrating these four pieces of waveform data.

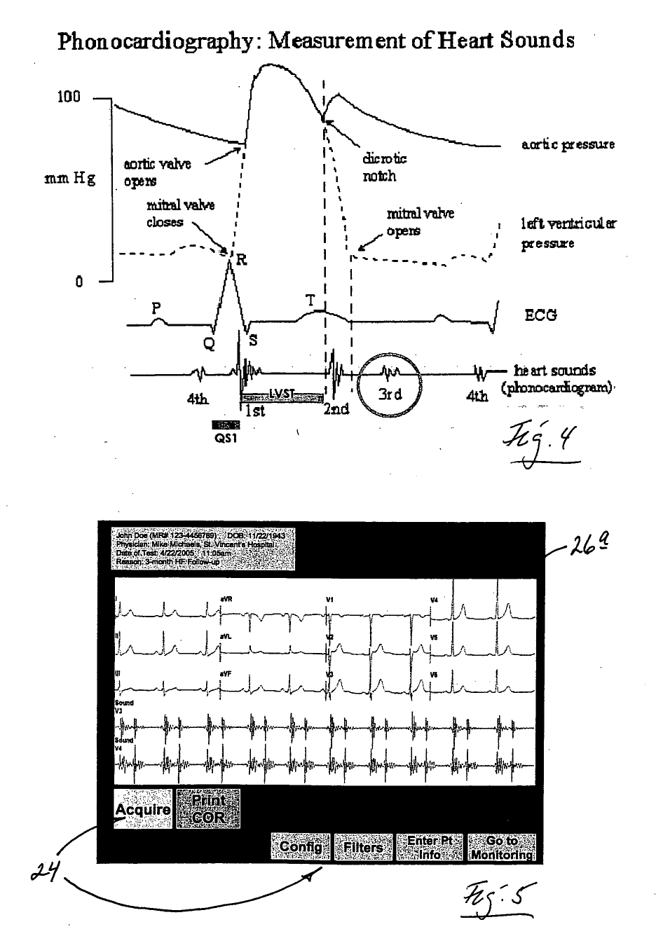

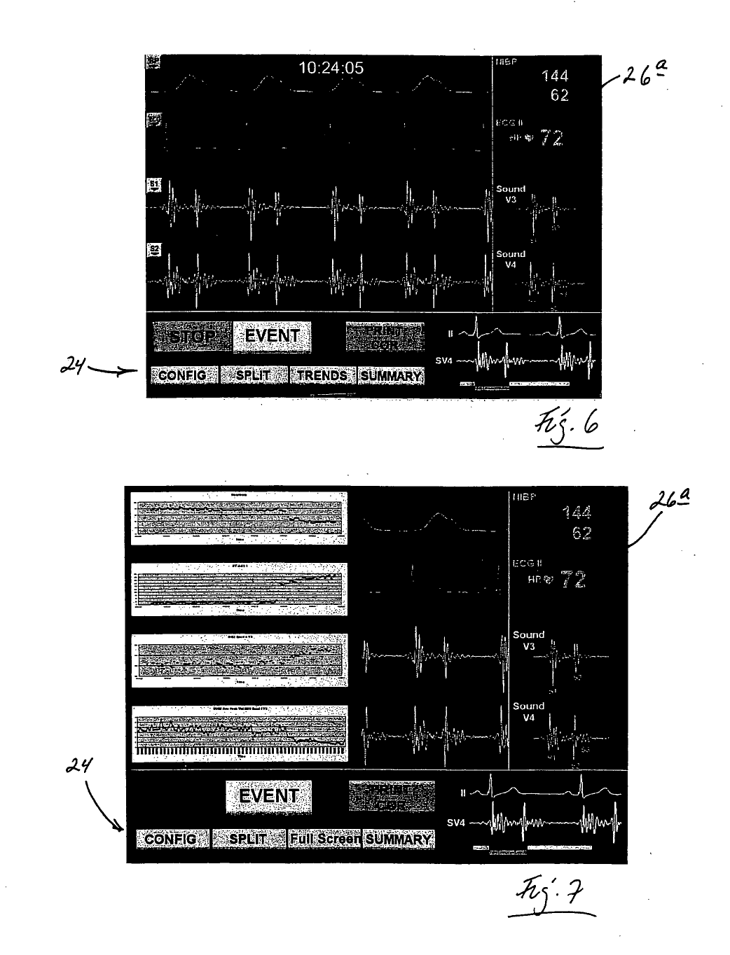

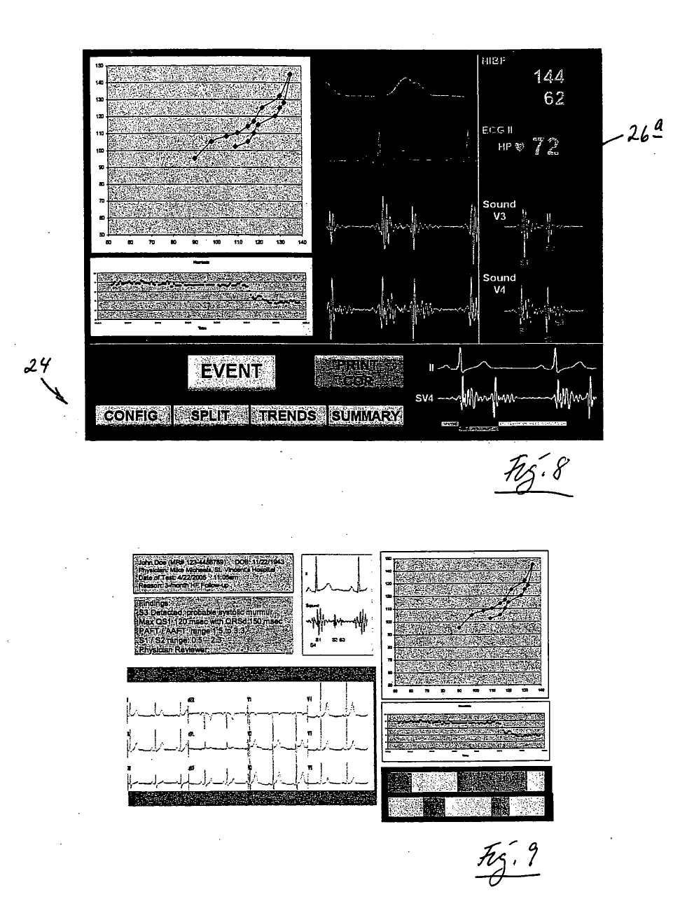

The other drawing figures included herein, namely, FIGS. 5-14, inclusive, will be discussed now along with an operational description of system 20.

With respect to performing an investigation of the cardio-functionality of a selected patient's heart, such as that of patient 22, the patient is suitably connected to system 20 through sensors such as those illustrated in FIGS. 1 and 2, which sensors will substantially always include ECG and heart-produced acoustic data sensors, along with any other additional data-collecting sensors desired by the system user. If the patient is to be coupled, so-to-speak, within a feedback loop of the type mentioned earlier pictured at 36 in FIGS. 1 and 2, that feedback loop relationship is established appropriately with the patient.

With these preparations completed, data collection begins over an extended, multiple-heartbeat period of time, with a user, via user-interface 24, (a) requesting that particular pieces of available input information be “brought into the system for processing”, and (b) also selecting whether and how particular pieces of information are to be displayed on the touchscreen in display output 26. As was mentioned earlier herein with respect to FIG. 2 in the drawings, blocks 62, 64 generally outline the input and display output selectability which is provided to a system user in accordance with the present invention. For example, such a user may request that a number of sequential heartbeats of input information derived from the sensors being presented in waveform style, and on a common time base for correlation purposes, on the screen of display output 26. The user might also request, or alternatively request, that only single waveform snippets of data be presented, with or without time-based correlation, on the display output.

FIGS. 5-8, inclusive, illustrate a relatively wide variety of display output presentations called for on touchscreen 26a, with these illustrations clearly showing the wide versatility of the present invention, system and methodology, to enable a system user to call for a very wide range of output information, including (a) graphical waveform information in plural-heartbeat, or abbreviated-heartbeat-snippet, forms, (b) time-based correlations of this waveform data including, of course, ECG and heart-produced acoustic data, (c) various forms of time-interval, bar-graph data, (d) various forms of numeric data, which may include numeric-ratio data where appropriate, and (e) textual data. Output display information also may include, as illustrated, appropriate textual data, and further may include several categories of computer-analyzed assessment and judgment-calling data. Specific illustrative details of what appears respectively on each of these screen displays are given above in the related descriptions of the drawings.

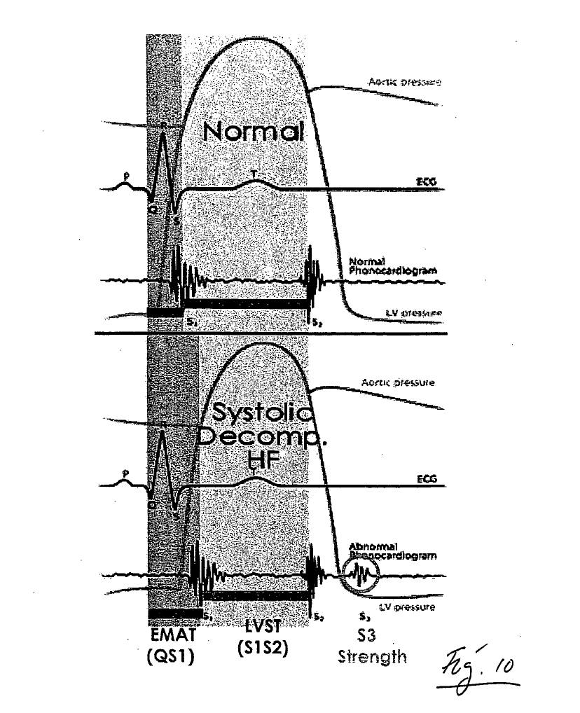

FIG. 9 presents a representative printed output display (as from printer 72 shown in FIG. 1) including the information generally described for this figure in the overall descriptions of the drawings above.

Those who are generally skilled in the relevant cardio-field art will, by looking at these representative display-screen views, immediately recognize the natures of the various data contents pictured there without any needed detailed and elaborate verbal descriptions.

When a system user calls for specialized use of analysis block 50 (see FIG. 2) which, as mentioned earlier herein, is referred to as prepared-intelligence, algorithmic, cardio-function analysis and interpretation structure, specially prepared analysis algorithms—the cardio-condition-assessing algorithmic software mentioned earlier herein—are appropriately applied to signal input data to prepare and present essentially “judgment-assessed” display output information. Such output information, on one level, may simply be limited to information from which a physician, or other clinician, can readily make a self-directed judgment call. On another level, this same general kind of information may include a condition-assessment “call”, or judgment, based upon computer analysis. On yet another level, such output information may produce an output data stream which is applied through control block 52 (see FIG. 2) to implement activity in a feedback loop such as feedback loop 36.

With respect to the operation of block 50, it should thus be noted that a relatively wide variety of useful, conventional, cardio-condition-assessing algorithms may be created, within the knowledge and skill of those generally skilled in the relevant art, for applying intelligent analysis-processing to data contained in input signal information in order to produce, from such analysis, high-level heart-condition assessments. The details of such algorithms form no part of the present invention, and, accordingly, are not discussed in any detail herein. In this regard, it is important to emphasize that a system user may call for, or not call, for the output of such computer-analysis information. Moreover, the user may clearly request the display output of such computer-performed assessments, along with other data elements, correlated or not, to support such computer-analysis results. Additionally, the user may request, based upon evidence presented in the display output information, that a control signal in a feedback loop be sent by the computer to adjust a device or therapy parameter which is associated with a subject patient.

Considering now the powerful trending capabilities of the present invention, it will be evident that the operation of the system and methodology of the invention function in a real-time data acquisition manner, and over an extended period of time, which involves the sequential collection of a continuum of heartbeat-produced information (i.e., plural heartbeats). In this context, and as was stated earlier herein, an extremely central and important concept of the present invention is implemented in the form of presenting cardio-function-condition trend behavior.

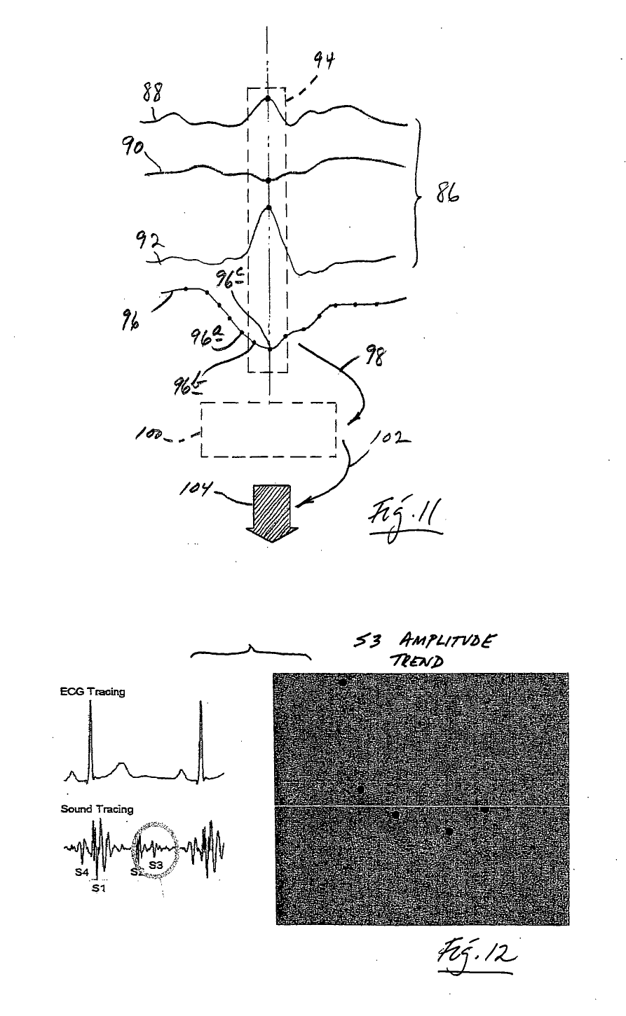

An example of this involves detecting the changing presence, absence, and amplitude of the so-called S3 heart sound over a period of time in which certain feedback “information” is being delivered to a subject patient. FIG. 10 in the drawings illustrates two (upper and lower) snippet-waveform-like display outputs which may be presented on a screen, such as screen 26a, to compare a condition where no S3 heart sound is present (the upper-illustrated waveform) with a condition where the S3 heart sound is indeed present with an identifiable certain amplitude (the lower-illustrated waveform). Over a selected period of time, feedback signals from computer 46 might be supplied in feedback loop 36 to control, in a staged, changing manner, the operation of a patient-installed pacemaker, so as to modify a patient's cardio behavior in a way which causes the undesirable S3 heart-sound, if found initially to be present, to vary in amplitude, and perhaps even to vanish, in response. By observing the S3 amplitude “trend” over this time period, via display-output-viewing of the associated, operative changes which occur in this amplitude (and thus in cardio-functionality) as a consequence of feedback activity, a system user can quickly determine a best-mode operation for the associated pacemaker, and can thereby significantly improve, almost immediately, the cardio-functionality of a patient's heart.

This activity may be practiced either manually by a system user, or, that user may “request” that system 20 automatically perform pacemaker-operation adjustment so as to maximize cardio-functionality in relation to observed condition-trend (S3-trend) behavior.

Here, it should be clearly understood that, while S3 trending in relation to pacemaker operation is now being specifically discussed, other aspects of cardio-functionality may also be constructively and correctively addressed on the basis of acquired, similar trending data. Also, feedback may take place in various appropriate other forms, such as in the form of drug-administration therapy.

Focusing attention at this point on drawing FIGS. 11-14, inclusive, and beginning with FIG. 11, here there are indicated, in an overall fashion, several important ways in which trend information, and output display presentation of such information, may be thus utilized. Highlighted by a bracket 86 in FIG. 11 are three time-base-correlated trend traces 88, 90, 92 which may be presented either as individuals, or, as just suggested, on the time correlation basis, on a display output screen, such as screen 26a, with specific points of interest, such as those shown at 88a, 90a, 92a, presented and perhaps even highlighted (as indicated by dashed block 94) for viewing and assessment by the system user. This kind of trend display, of course, offers the opportunity for a system user to understand relatively quickly important cardio-functionality conditions existing within a subject patient's heart.

Another trend-based trace is shown at 96, with data points taken over time to indicate trend behavior of a particular condition shown, for example, at 96a, 96b, 96c.

Previously mentioned dashed block 94 represents output information which is delivered to a system user without any necessarily reported computer analysis. If desired, and as such as illustrated very generally by curved arrow 98 in FIG. 11, a system user may call for the same kind of visual output accompanied by a computer-analyzed data assessment, or judgment, which is represented in FIG. 11 by dashed block 100. An illustration of such a textual-based computer assessment is pictured near the upper left-hand corner of FIG. 9 in the drawings.

Further, a system user may instruct the system computer, as generally illustrated by curved arrow 102 in FIG. 11, to send a control output data stream 104 into feedback loop 36 to effect some sort of parameter change in the application of an administered drug therapy, or in the operation of a device, such as a pacemaker, or a treadmill.

It will thus be evident that the system and methodology of this invention indeed provide a highly versatile and flexible approach to acquiring, analyzing, presenting and utilizing cardio-relevant data acquired in real time from a subject patient, with the important opportunity given to utilize trending information regarding cardio-functionality to effect corrective controls. For example, if a trend illustrates the possibility for changing certain control parameters so as to minimize, or eliminate entirely, a negative cardio condition, such as the presence of the S3 heart sound, the system of this invention offers a system user the opportunity to observe just what to do in order to bring this condition of improved cardio-functionality about.

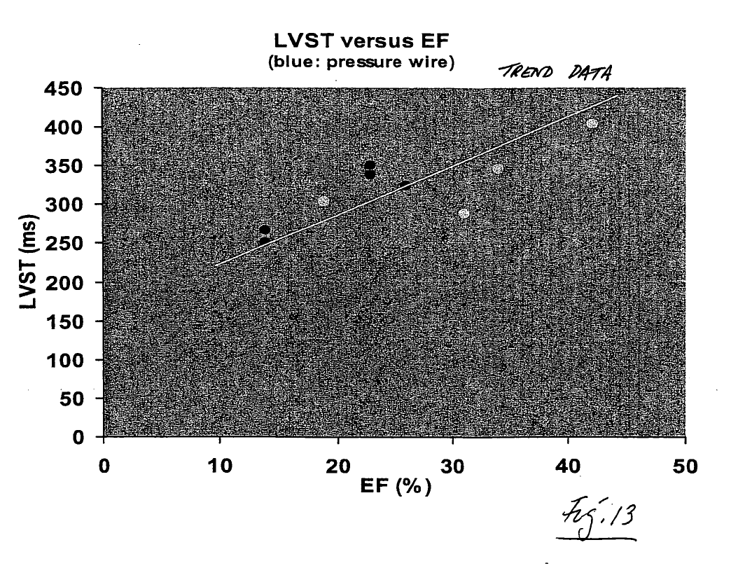

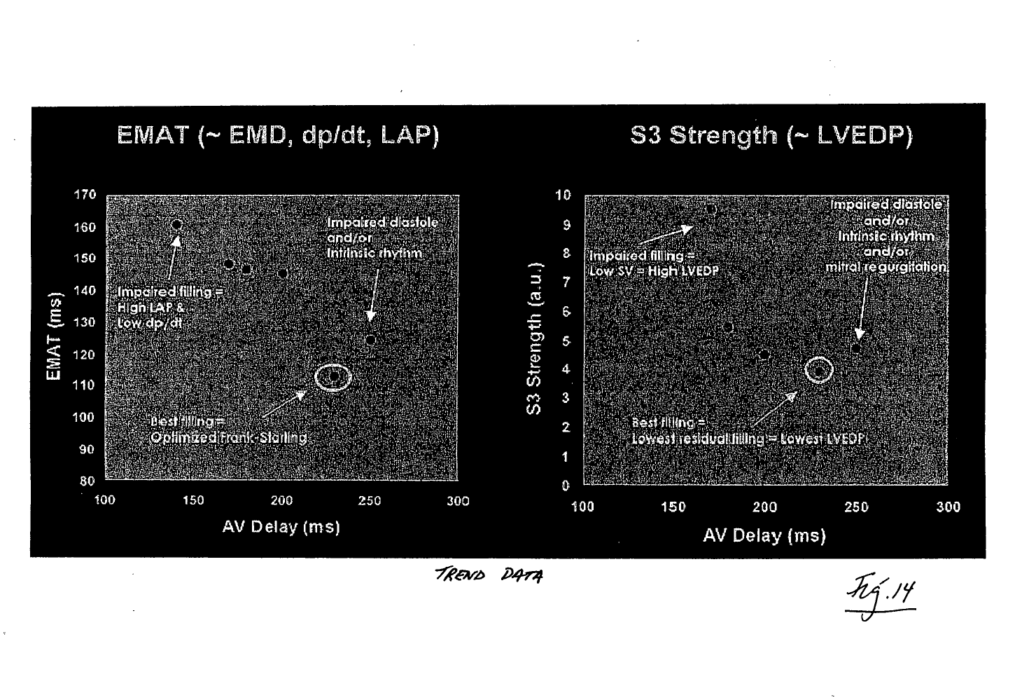

FIGS. 12, 13 and 14 illustrate different, self explaining presentations of different kinds of trend-observed patient behavior, with FIG. 12 illustrating, generally speaking, a trend involving amplitude changes over time of heart-sound S3 amplitude (discussed in certain detail above), with FIG. 13 illustrating a trend relationship between LVST and EF (ejection fraction), and with FIG. 14 illustrating another pair of trends clearly identified in this figure.

From all of the discussion above, taken in conjunction with the several drawing figures, it should now be evident how the present invention offers a significant advance in the relevant art.

From the discussion presented above, one way of characterizing the advanced methodology of the invention is to describe it as a method for gathering, handling, observing and presenting cardio-function data from a selected subject patient, and for utilizing that data to effect constructive and corrective, data-trend-based, cardio-function intervention, including the steps of: (a) in real time, gathering cardio-relevant cardio-functionality data from a subject patient including, over time, selected-category, cardio-functionality trend data; (b) utilizing such trend data in an implemented feedback manner to effect real-time changes in the subject patient's cardio-functionality as evidenced by that trend data; and (c) while so implementing the mentioned feedback manner of utilization, continuing to gather and observe the same-category trend data so as to achieve, through utilization feedback, and related constructive and corrective intervention, improved cardio-functionality in relation to the associated trend data.

Another way to describe the invention methodology is to view it as being aimed at the same, just-above-mentioned, overall practice including the steps of: (a) gathering, over a selected period of time, real-time, cardio-relevant, patient-specific data, including ECG data and heart-produced acoustic data: (b) computer-processing such gathered data, including selectively applying cardio-condition-assessing algorithmic software to the data; (c) selectively display-presenting input and computer-processed data, along with, as desired, selected, algorithmically-assessed cardio-condition data, in forms including at least one of the categories of (1) plural-heartbeat waveforms, (2) single-heartbeat waveform snippets, (3) cardio-condition trend data, (4) numeric data, and (5) textual data, with or without accompanying judgment comment based upon computer-implemented assessment of such data; (d) selectively enabling manual, or computer-directed-automatic, constructive and corrective feedback-intervention relative to a subject patient based upon selected trend data so as to improve a selected-trend aspect of the subject patient's cardio-functionality by producing observable, operative changes in such functionality; and (e) confirming such improvement by continuing to gather, present and observe relevant cardio-condition selected-trend data.

Accordingly, while a preferred embodiment, and a preferred manner of practicing, the present invention have been described and illustrated herein, and while certain modified practices have been suggested, it is appreciated that other variations and modifications may be made by those skilled in the art, without such variations and modifications departing from the spirit of the invention, and with all such variations and modification therefore clearly coming within the scope of the present invention.

We claim:

1. A computer-based cafeteria system, accessible through an included user interface, for gathering, handling, observing and presenting cardio-function data from a selected subject patient, and for utilizing that data to effect constructive and corrective, data-trend-based, cardio-function intervention, said system comprising
structure operatively connectable to, for collecting from, such a patient, in electrical signal form, relevant, plural-heartbeat, cardio-function data, including ECG and heart-produced acoustic data,
computer data-processing apparatus operatively connected to said collecting structure for receiving, and in a system-user-selectable/directable manner, inter-associating, through processing, selected elements of such collected data so as to offer, via user-selected printed and/or screen-presented display output, made available in image forms including (a) graphical, numeric, and textual styles, and (b) blends of such styles, an opportunity to assess the presence and character of a user-reviewable, cardio-function condition of the selected subject patient's heart, including the detectable presence of cardio-function-condition trend behavior of that heart, useable to effect/implement constructive and corrective cardio-function intervention, and
display output structure operatively connected to said data-processing apparatus, activatible by a user to present an assessment-relevant, singular, or selectively multi-faceted, cardio-function-condition display (a) of such inter-associated data, (b) of selected elements thereof, and (c) of such detected cardio-function-condition trend behavior.

2. The system of claim 1, wherein said display output structure includes at least one of (a) printing structure, and (b) electronic screen-display structure.

3. The system of claim 1 which forms part of a feedback loop that also includes (a) the selected subject patient, and (b) a cardio-function control-parameter changer which, in this loop, is structured to act, on the basis of offered display output, as a link to constructive and corrective intervention relative to, and in response to, a system-observed, negative cardio function of the selected patient's heart, as reflected in detected cardio-function-condition trend behavior

4. The system of claim 1, wherein said computer data-processing apparatus is constructed to offer display output including, as illustrations, (a) direct waveform display of collected ECG and heart-produced acoustic data, and (b) selected waveform display snippets of ECG and heart-produced acoustic waveform data.

5. The system of claim 4 which further includes prepared-intelligence, algorithmic, cardio-function analysis and interpretation structure operatively associated with, and accessible by, said data-processing apparatus, and wherein said data-processing apparatus is further constructed, via use of said analysis and interpretation structure, to offer display output including (a) cardio-function correlation data, (b) external-analysis-readied cardio-function data, (b) computer-analysis-suggested cardio-function data, (c) computer-analyzed cardio-function data, (d) poised-for-computer-action, computer-analyzed, cardio-function-condition data, and (e) computer-analyzed, cardio-function-condition, trend-behavior data.

6. The system of claim 5, wherein the (a), (b), and (c) types of data mentioned therein are based upon observed, cardio-function trend behavior.

7. The system of claim 5, wherein a heart-related control-parameter changer is operatively connected to a subject patient, and the mentioned trend-behavior data is based upon control-parameter parameter changes applied over time to the patient.

8. The system of claim 7 which further comprises an operative link between said computer data-processing apparatus and the mentioned control-parameter changer, and the computer data-processing apparatus is further constructed to operate the control-parameter changer in relation to the computer-analyzed, cardio-function-condition, trend-behavior data.

9. A method for gathering, handling, observing and presenting cardio-function data from a selected subject patient, and for utilizing that data to effect constructive and corrective, data-trend-based, cardio-function intervention, said method comprising
in real time, gathering cardio-relevant cardio-functionality data from a subject patient including, over time, selected-category, cardio-functionality trend data,
utilizing such trend data in an implemented feedback manner to effect real-time changes in the subject patient's cardio-functionality as evidenced by that trend data, and
while so implementing the mentioned feedback manner of utilization, continuing to gather and observe the same-category trend data so as to achieve, through utilization feedback, and related constructive and corrective intervention, improved cardio-functionality in relation to the associated trend data.

10. The method of claim 9, wherein said utilizing is performed in at least one of the manners including (a) manually, and (b) automatically under computer control.

11. A method for gathering, handling, observing and presenting cardio-function data from a selected subject patient, and for utilizing that data to effect constructive and corrective, data-trend-based, cardio-function intervention, said method comprising
gathering, over a selected period of time, real-time, cardio-relevant, patient-specific data, including ECG data and heart-produced acoustic data,
computer-processing such gathered data, including selectively applying cardio-condition-assessing algorithmic software to the data,
selectively display-presenting input and computer-processed data, along with, as desired, selected, algorithmically-assessed cardio-condition data, in forms including at least one of the categories of (a) plural-heartbeat waveforms, (b) single-heartbeat waveform snippets, (c) cardio-condition trend data, (d) numeric data, and (e) textual data, with or without accompanying judgment comment based upon computer-implemented assessment of such data,
selectively enabling manual, or computer-directed-automatic, constructive and corrective feedback-intervention relative to a subject patient based upon selected trend data so as to improve a selected-trend aspect of the subject patient's cardio-functionality by producing observable changes in such functionality, and
confirming such improvement by continuing to gather, present and observe relevant cardio-condition selected-trend data.
